(19) United States
(12) Patent Application Publication
(10) Pub. No.: US 2025/0213448 A1
(43) Pub. Date: Jul. 3, 2025

(54) COMPOSITION FOR PROTECTING SKIN CONTAINING A THYMOL ESTER-BASED COMPOUND

(71) Applicant: Amorepacific Corporation, Seoul (KR)

(72) Inventors: Hyunjung CHOI, Yongin-si (KR); Hye Won NA, Yongin-si (KR); Kyuhan KIM, Yongin-si (KR); Hyoung June KIM, Yongin-si (KR); Eun Jeong CHOI, Yongin-si (KR)

(73) Assignee: Amorepacific Corporation, Seoul (KR)

(21) Appl. No.: 18/990,489

(22) Filed: Dec. 20, 2024

(30) Foreign Application Priority Data
Jan. 2, 2024 (KR) 10-2024-0000274

(51) Int. Cl. A61K 8/37; A61Q 19/00; A61Q 19/08

(52) U.S. Cl. CPC A61K 8/375; A61Q 19/004; A61Q 19/08

(57) ABSTRACT

A composition for protecting skin, including 3,4,5-trimethoxycinnamate thymol ester, a stereoisomer, pharmaceutically acceptable salt, hydrate, or solvate thereof as an active ingredient, is disclosed. The composition can inhibit or prevent skin damage due to ultraviolet rays, external environment, or oxidative stress by increasing the expression of a DNA repair-related gene in skin cells photoaged by ultraviolet rays.

### CROSS REFERENCE TO RELATED APPLICATION

This application claims priority to Korea Patent Application No. 10-2024-0000274 filed on Jan. 2, 2024, the entire contents of which are incorporated herein by reference.

### BACKGROUND OF THE INVENTION

### Field of the Invention

Disclosed herein is a composition for protecting skin, including 3,4,5-trimethoxycinnamate thymol ester or a stereoisomer, pharmaceutically acceptable salt, hydrate, or solvate thereof as an active ingredient.

### Description of the Related Art

Since the skin is the largest tissue surrounding the outermost part of the human body, it is affected more than anything else by the external environment. In particular, harmful light, such as ultraviolet rays, which cause oxidative stress, and environmental pollutants, such as fine dust, directly act on the skin cells that make up the skin, damaging the genome such as DNA. If this damaged DNA is not properly repaired, cell function abnormalities, cell death, or diseases such as cancer may occur.

There are various methods for repairing DNA damage depending on the cause and degree of damage, and many genes are involved in this. As the skin ages, the expression of genes involved in DNA damage decreases, greatly reducing the ability to repair DNA. As a result, skin damage can occur even with a small exposure to a harmful environment.

As people's lifespan increases and environmental pollution worsens, factors that cause skin damage continue to accumulate, so it is a very necessary technology to enhance the biological ability to repair DNA damage. However, the development of skin damage prevention technology that can prevent and inhibit skin damage without other side effects is insufficient.

### SUMMARY OF THE INVENTION

### Technical Problem

In one aspect of the present invention, it is intended to provide a composition having a skin-protecting effect by including 3,4,5-trimethoxycinnamate thymol ester as an active ingredient.

### Technical Solution

In one aspect, the present disclosure provides a composition for protecting skin, including 3,4,5-trimethoxycinnamate thymol ester or a stereoisomer, pharmaceutically acceptable salt, hydrate, or solvate thereof as an active ingredient.

### Advantageous Effects

In one aspect, the composition according to the present disclosure can inhibit or prevent skin damage or aging.

In one aspect, the composition according to the present disclosure can inhibit or prevent skin damage due to photoaging.

In one aspect, the composition according to the present disclosure can increase the expression of a gene that repairs DNA damage.

In one aspect, the composition according to the present disclosure can increase the expression of a gene that repairs DNA damaged by photoaging.

In one aspect, the composition according to the present disclosure can inhibit or prevent skin damage due to ultraviolet rays, external environment, or oxidative stress by increasing the expression of a DNA repair-related gene in skin cells photoaged by ultraviolet rays.

### BRIEF DESCRIPTION OF THE DRAWINGS

FIGS. 1 and 2 show the results of observing whether or not aging genes are expressed according to one embodiment of the present disclosure.

FIGS. 3 to 5 show the results of observing whether or not DNA repair genes are expressed according to one embodiment of the present disclosure, respectively.

### DETAILED DESCRIPTION OF THE INVENTION

Hereinafter, the present invention will be described in more detail by way of the following examples. However, the following examples are provided only for illustrative purposes to aid understanding of the present invention, and the scope and range of the present invention are not limited thereto.

In exemplary embodiments of the present invention, there is provided a composition for protecting skin, including 3,4,5-trimethoxycinnamate thymol ester, a stereoisomer, pharmaceutically acceptable salt, hydrate, or solvate thereof as an active ingredient.

In another exemplary embodiment of the present invention, there is provided a method for protecting skin, the method comprising administering a composition including 3,4,5-trimethoxycinnamate thymol ester, a stereoisomer, pharmaceutically acceptable salt, hydrate, or solvate thereof as an active ingredient.

In yet another exemplary embodiment of the present invention, there is provided a use of 3,4,5-trimethoxycinnamate thymol ester, a stereoisomer, pharmaceutically acceptable salt, hydrate, or solvate thereof for preparing a composition for protecting skin.

In still another exemplary embodiment of the present invention, there is provided a non-therapeutic use of 3,4,5-trimethoxycinnamate thymol ester, a stereoisomer, pharmaceutically acceptable salt, hydrate, or solvate thereof for protecting skin.

In still another exemplary embodiment of the present invention, there is provided 3,4,5-trimethoxycinnamate thymol ester, a stereoisomer, pharmaceutically acceptable salt, hydrate, or solvate thereof for protecting skin.

In one embodiment, the skin protection may be inhibition or prevention of skin damage.

In one embodiment, the skin protection may be improvement of skin self-regenerative power.

In one embodiment, the skin protection may be DNA damage repair.

In one embodiment, the 3,4,5-trimethoxycinnamate thymol ester is (5-methyl-2-propan-2-ylphenyl) (E)-3-(3,4,5-trimethoxyphenyl) prop-2-enoate, which may be represented by Chemical Formula 1 below:

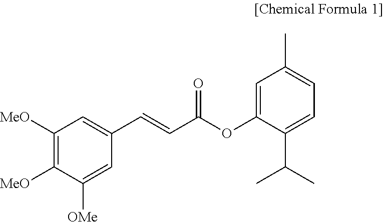 [SMILES: COc1cc(/C=C/C(=O)Oc2cc(C)ccc2C(C)C)cc(OC)c1OC]

As used herein, “isomer” includes in particular optical isomers (e.g., essentially pure enantiomers, essentially pure diastereomers or mixtures thereof), as well as conformation isomers (i.e., isomers which differ only in the angles of one or more chemical bonds), position isomers (in particular, tautomers) or geometric isomers (e.g., cis-trans isomers).

As used herein, the term “essentially pure”, for example, when used in connection with an enantiomer or diastereomer, means that a specific compound, for example an enantiomer or diastereomer, is present in an amount of at least about 90%, preferably at least about 95%, more preferably at least about 97% or at least about 98%, even more preferably at least about 99%, and even more preferably at least about 99.5% (w/w).

As used herein, the term “pharmaceutically acceptable” means that it can be or has been approved by the government or equivalent regulatory body as being suitable for use in animals, and more particularly, in humans, by avoiding significant toxic effects when used in conventional medicinal dosages, or is listed in a pharmacopoeia or recognized in other general pharmacopoeias.

As used herein, the term “pharmaceutically acceptable salt” means a salt according to one aspect of the present invention that is pharmaceutically acceptable and has the desired pharmacological activity of the parent compound. The salt may include (1) an acid addition salt formed of inorganic acid such as hydrochloric acid, hydrobromic acid, sulfuric acid, nitric acid, phosphoric acid, etc.; or formed of organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfate, gluconic acid, glutamic acid, hydroxynaphthoic acid, salicylic acid, stearic acid, muconic acid.; or (2) a salt formed when an acidic proton present in the parent compound is substituted.

As used herein, the term “hydrate” refers to a compound to which water is bound, and is a broad concept that includes an inclusion compound with no chemical bonding force between water and the compound.

As used herein, the term “solvate” refers to a high-order compound formed between a molecule or ion of a solute and a molecule or ion of a solvent.

In one embodiment, the composition may be for inhibiting or preventing skin damage.

In one embodiment, the composition may be for improving skin self-regenerative power.

In one embodiment, the composition may be for repairing DNA damage.

In one embodiment, the damage may be damage caused by at least one selected from the group consisting of oxidative stress, alkali, drugs, aging, and ultraviolet rays.

In one embodiment, the ultraviolet rays may be at least one of UVA and UVB.

In one embodiment, the composition may increase the expression of DNA repair genes.

In one embodiment, the DNA repair genes may include at least one selected from the group consisting of APEX1, XPA, and RPA1.

The APEX1 is a gene involved in base excision repair, the XPA is a gene involved in nucleotide excision repair, and the RPA1 is a gene involved in mismatch repair. The base excision repair is mainly involved in DNA damage caused by oxidative stress or alkali, and the nucleotide excision repair is involved in DNA damage caused by ultraviolet rays or anticancer agents. Finally, the mismatch repair is involved in repairing DNA damage that may occur when cells are replicated.

In one embodiment, the composition can inhibit the expression of aging genes.

In one embodiment, the aging genes may include one or more of P21 and P16.

In one embodiment, the concentration of the active ingredient may be 0.1 to 100 g/L based on the total volume of the composition.

For example, the concentration of the active ingredient may be 0.1 g/L or more, 0.5 g/L or more, 1 g/L or more, 5 g/L or more, 10 g/L or more, 20 g/L or more, 30 g/L or more, 40 g/L or more, 50 g/L or more, 60 g/L or more, 70 g/L or more, 80 g/L or more, or 90 g/L or more, and may be 100 g/L or less, 90 g/L or less, 80 g/L or less, 70 g/L or less, 60 g/L or less, 50 g/L or less, 40 g/L or less, 30 g/L or less, 20 g/L or less, 10 g/L or less, 5 g/L or less, 1 g/L or less, or 0.5 g/L or less, based on the total volume of the composition.

In one embodiment, the composition may be a skin external application composition.

In one embodiment, the composition may be a non-therapeutic transdermal composition.

The skin external application or transdermal composition is a generic term that may include anything that is applied externally to the skin, and may include various types of cosmetics, pharmaceuticals, etc.

In one embodiment, the composition may be a cosmetic composition.

The external form of the cosmetic composition contains a cosmetically or dermatologically acceptable medium or base. It may be provided in any formulation suitable for topical application, for example, in the form of a solution, a gel, a solid, a pasty anhydrous product, an emulsion obtained by dispersing an oil phase in an aqueous phase, a suspension, a microemulsion, a microcapsule, a microgranule, or ionic (liposome) and nonionic vesicular dispersion, or in the form of a cream, a skin, a lotion, a powder, an ointment, a spray, or a concealer stick. These compositions can be prepared according to conventional methods in the art. The composition according to the present disclosure may also be used in the form of a foam or in the form of an aerosol composition further containing a compressed propellant.

The cosmetic composition according to one embodiment of the present disclosure is not particularly limited in its formulation, and may be formulated into cosmetics such as a softening toner, an astringent toner, a nourishing toner, a nourishing cream, a massage cream, an essence, an eye cream, an eye essence, a cleansing cream, a cleansing foam, a cleansing water, a pack, a powder, a body lotion, a body cream, a body oil, and a body essence.

When the formulation of the cosmetic composition of the present disclosure is a paste, a cream, or a gel, animal fiber, plant fiber, wax, paraffin, starch, tragacanth, a cellulose derivative, polyethylene glycol, silicone, bentonite, silica, talc, or zinc oxide may be used as a carrier component.

When the formulation of the cosmetic composition of the present disclosure is a powder or spray, lactose, talc, silica, aluminum hydroxide, calcium silicate or polyamide powder may be used as a carrier component, and in particular, in the case of a spray, a propellant such as chlorofluorohydrocarbon, propane/butane or dimethyl ether may be additionally included.

When the formulation of the cosmetic composition of the present disclosure is a solution or an emulsion, a solvent, a solvating agent or an emulsifying agent is used as a carrier component, and the examples include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or a fatty acid ester of sorbitan.

When the formulation of the cosmetic composition of the present disclosure is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used as a carrier component.

When the formulation of the cosmetic composition of the present disclosure is a surfactant-containing cleansing, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, aliphatic alcohol, fatty acid glyceride, fatty acid diethanolamide, vegetable oil, linolin derivative, ethoxylated glycerol fatty acid ester, etc. may be used as a carrier ingredient.

The cosmetic composition of the present disclosure may further include a functional additive and a component included in a general cosmetic composition in addition to the above-described active ingredients. The functional additive may include a component selected from the group consisting of water-soluble vitamins, oil-soluble vitamins, polymer peptides, polymer polysaccharides, sphingo lipids, and seaweed extract.

In the cosmetic composition of the present disclosure, in addition to the above functional additive, a component included in a general cosmetic composition may be blended as necessary. In addition, other blending components, such as oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulation promoters, cooling agents, anhydrotics, purified water, etc., may be additionally included.

In one embodiment, the composition may be a food composition.

In one embodiment, the composition may be a non-therapeutic oral composition.

The food composition or non-therapeutic oral composition according to the present disclosure may be a liquid or solid formulation, and may be a tablet, a capsule, a soft capsule, a pill, a granule, a beverage (drinking agent), a diet bar, a chocolate, a caramel formulation, or a confectionery formulation, but the formulation is not particularly limited.

In addition to the above active ingredients, the food composition or non-therapeutic oral composition according to the present disclosure may further include excipients, sugars, flavors, pigments, fats, proteins, etc., as needed.

In one embodiment, the 3,4,5-trimethoxycinnamate thymol ester, the stereoisomer, pharmaceutically acceptable salt, hydrate, or solvate thereof may be applied or ingested at an application or ingestion amount of 0.01 to 10 mg/kg/day.

For example, the application or ingestion amount may be 0.01 mg/kg/day or more, 0.1 mg/kg/day or more, 0.5 mg/kg/day or more, 1 mg/kg/day or more, 3 mg/kg/day or more, 5 mg/kg/day or more, 7 mg/kg/day or more, or 9 mg/kg/day or more, and may be 10 mg/kg/day or less, 8 mg/kg/day or less, 6 mg/kg/day or less, 4 mg/kg/day or less, 2 mg/kg/day or less, 1 mg/kg/day or less, 0.5 mg/kg/day or less, 0.1 mg/kg/day or less, or 0.05 mg/kg/day or less.

In one embodiment, the composition may be a pharmaceutical composition.

The pharmaceutical composition according to one embodiment of the present invention may additionally contain pharmaceutical adjuvants such as preservatives, stabilizers, hydrating agents or emulsifying agents, salts for osmotic pressure control, and/or buffers, and other therapeutically useful substances, and may be formulated into various oral or parenteral dosage forms according to conventional methods.

The oral dosage forms include, for example, tablets, pills, hard and soft capsules, solutions, suspensions, emulsions, syrups, dusts, powders, fine granules, granules, pellets, etc., and these formulations may contain, in addition to the active ingredient, a surfactant, a diluent (e.g., lactose, dextrose, sucrose, mannitol, sorbitol, cellulose, and glycine), and a lubricant (e.g., silica, talc, stearic acid and its magnesium or calcium salts, and polyethylene glycol). The tablet may also contain a binder such as magnesium aluminum silicate, starch paste, gelatin, tragacanth, methylcellulose, sodium carboxymethylcellulose, and polyvinylpyrrolidine, and may optionally contain pharmaceutical additives such as a disintegrant, an absorbent, a coloring agent, a flavoring agent, and a sweetener, such as starch, agar, alginic acid or a sodium salt thereof. The tablet may be manufactured by a conventional mixing, granulating, or coating method.

In addition, the parenteral dosage form may be a transdermal dosage formulation, for example, a formulation such as an injection, a drop, an ointment, a lotion, a gel, a cream, a spray, a suspension, an emulsion, a suppository, a patch, etc., but is not limited thereto.

In one embodiment, the 3,4,5-trimethoxycinnamate thymol ester, the stereoisomer, pharmaceutically acceptable salt, hydrate, or solvate thereof may be administered at a dosage of 0.01 to 30 mg/kg/day.

For example, the dosage may be 0.01 mg/kg/day or more, 0.1 mg/kg/day or more, 1 mg/kg/day or more, 5 mg/kg/day or more, and may be 10 mg/kg/day or less, 5 mg/kg/day or less, 1 mg/kg/day or less, 0.1 mg/kg/day or less, or 0.05 mg/kg/day or less.

Hereinafter, the present invention will be described in more detail by way of the following examples. However, the following examples are provided only for illustrative purposes to aid understanding of the present invention, and the scope and range of the present invention are not limited thereto.

### EXAMPLES

### Preparation of 3,4,5-trimethoxycinnamate thymol ester

Thymol trimethoxycinnamate thymol ester with CAS number 504394-57-4 (Melasolv) was obtained from COSMANN Co., Ltd. (Hwaseong-si, Gyeonggi-do, Korea).

### Experimental Example

### Experimental Example 1—Damage Inhibition Experiment Using Normal Human Melanocyte

Melanocytes isolated from normal human skin (Normal human epidermal melanocytes) were treated with ultraviolet B (312 nm) at 20 mJ each, once a day, for two days, and cultured for one month. Melasolv was dissolved in DMSO at 1000×, irradiated with ultraviolet rays, and then immediately placed in a culture medium at 1 and 5 ppm and treated. The cells were grown in a 37° C., 5% CO₂ incubator while changing the culture medium containing the experimental material every two days. After the experiment was completed, the cells were dissolved in TRIzol and collected, and total RNA was isolated. Then, cDNA was synthesized therefrom and real-time PCR was performed to compare the relative expression levels of each gene. The RNA was quantified and the same amount of RNA was added to each experimental group, and the expression of each gene was corrected by the expression of GAPDH, a house-keeping gene, to compare the relative expression levels.

The gene primers used were purchased from APPLIED BIOSYSTEMS as follows:

APEX1 (Hs00172396_m1), XPA (Hs00902270_m1), RPA1 (Hs00161419_m1), GAPDH (Hs02786624_g1), p21 (Hs00355782_m1), p16 (Hs00923894_m1)

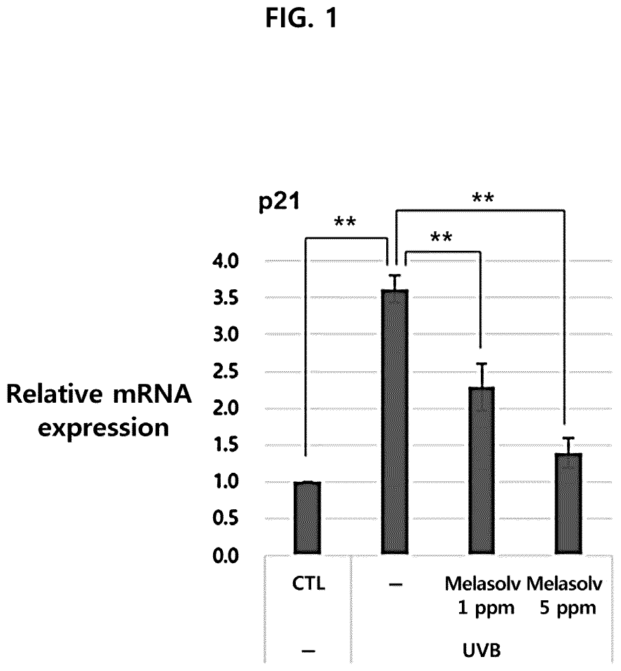

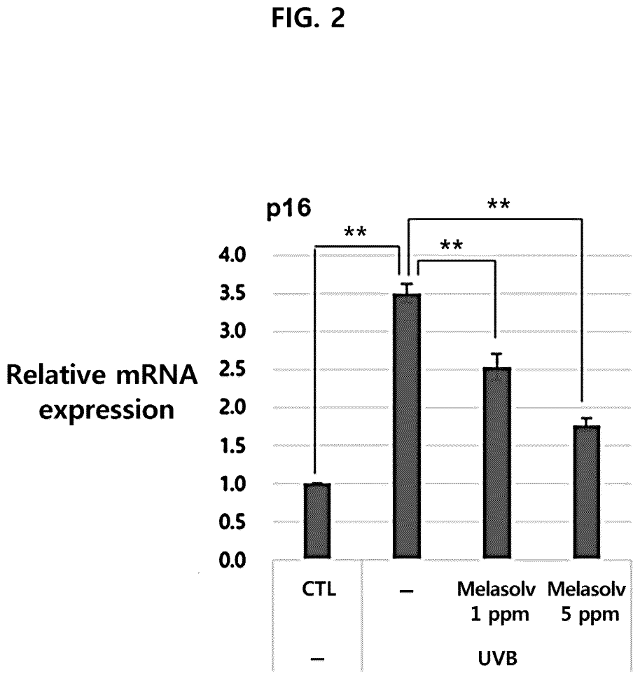

First, as shown in FIGS. 1 and 2, it could be confirmed that skin cells (melanocytes) treated with ultraviolet rays and cultured for a month were aged due to increased aging genes p16 and p21.

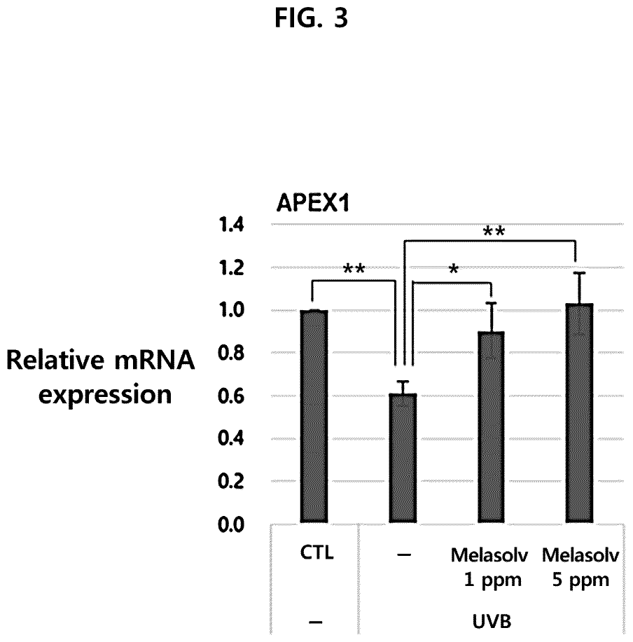

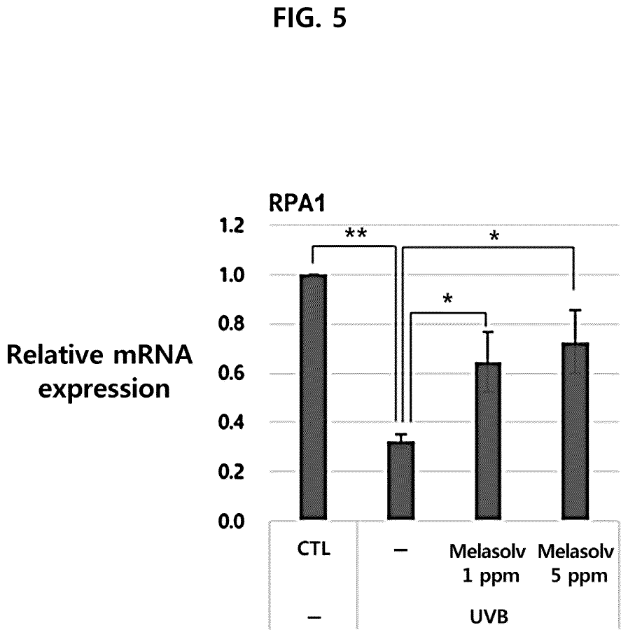

Thereafter, as shown in FIGS. 3 to 5, the expression of three representative genes involved in DNA repair was confirmed. At this time, it could be confirmed that all kinds of DNA repair genes were statistically significantly reduced by photoaging.

However, it could be confirmed that when cells were irradiated with ultraviolet rays and treated with Melasolv at 1 and 5 ppm, the expression of aging genes was inhibited in a concentration-dependent manner (see FIGS. 1 and 2), and the expression of DNA repair genes that had decreased due to photoaging was increased (see FIGS. 3 to 5).

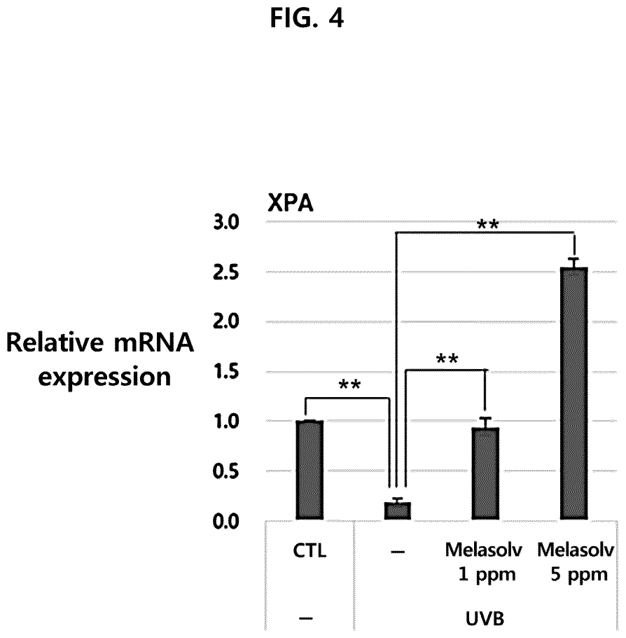

In particular, it could be confirmed that the expression of the XPA gene of FIG. 4 was increased even higher than that of the control group by 5 ppm of Melasolv, thereby remarkably improving the DNA repair ability. This is a result showing that Melasolv can prevent and inhibit skin damage by increasing the ability of cells to repair all kinds of DNA damage caused by oxidative stress, alkali, drug, ultraviolet rays, and aging. (*P<0.05, **P<0.01)

What is claimed is:

1. A method for protecting skin, the method comprising administering to a subject in need thereof an effective amount of a composition including 3,4,5-trimethoxycinnamate thymol ester, a stereoisomer, pharmaceutically acceptable salt, hydrate, or solvate thereof as an active ingredient.

2. The method according to claim 1, wherein the protecting skin comprises inhibiting or preventing skin damage.

3. The method according to claim 1, wherein the protecting skin comprises improving skin self-regenerative power.

4. The method according to claim 1, wherein the protecting skin comprises repairing DNA damage.

5. The method according to claim 2, wherein the damage is damage caused by at least one selected from the group consisting of oxidative stress, alkali, drugs, aging, and ultraviolet rays.

6. The method according to claim 5, wherein the ultraviolet rays are at least one of UVA and UVB.

7. The method according to claim 1, wherein the protecting skin comprises increasing the expression of DNA repair genes.

8. The method according to claim 7, wherein the DNA repair genes include at least one selected from the group consisting of APEX1, XPA, and RPA1.

9. The method according to claim 1, wherein the protecting skin comprises inhibiting the expression of aging genes.

10. The method according to claim 9, wherein the aging genes include one or more of P21 and P16.

11. The method according to claim 1, wherein the concentration of the active ingredient is 0.1 to 100 g/L based on the total volume of the composition.

12. The method according to claim 1, wherein the composition is a skin external application composition.

13. The method according to claim 1, wherein the composition is a cosmetic composition.

14. The method according to claim 1, wherein the composition is a food composition.

15. The method according to claim 1, wherein the composition is a pharmaceutical composition.

16. The method according to claim 1, wherein the 3,4,5-trimethoxycinnamate thymol ester, the stereoisomer, pharmaceutically acceptable salt, hydrate, or solvate thereof is administered at a dosage of 0.01 to 30 mg/kg/day.
